(19) United States
(12) Patent Application Publication
(10) Pub. No.: US 2010/0104520 A1
(43) Pub. Date: Apr. 29, 2010

(54) COSMETIC OR DERMATOLOGICAL COMPOSITION IN THE FORM OF AN OIL-IN-WATER OR WATER-IN-OIL-IN-WATER EMULSION COMPRISING A HEAT-INDUCED GELLING POLYMER, A WATER-MISCIBLE VOLATILE ORGANIC SOLVENT AND AN ORGANIC UV-SCREENING AGENT

(76) Inventor: Didier Candau, Bievres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/373,498

(22) PCT Filed: Jun. 21, 2007
(86) PCT No.: PCT/EP07/56199

§ 371 Date: Oct. 20, 2009

Related U.S. Application Data
(60) Provisional application No. 60/833,244, filed on Jul. 26, 2006.

(30) Foreign Application Priority Data
Jul. 13, 2006 (FR) 0652977

(51) Int. Cl. A61K 8/36; A61Q 17/04

(52) U.S. Cl. 424/59

(57) ABSTRACT

Cosmetic or dermatological composition in the form of an oil-in-water or water-in-oil-in-water emulsion comprising a heat-induced gelling polymer, a water-miscible volatile organic solvent and an organic UV-screening agent The present invention relates to a cosmetic or dermatological composition in the form of an oil-in-water emulsion or of water-in-oil-in-water multiple emulsion, characterized in that it comprises, in a physiologically acceptable medium: a) at least one continuous aqueous phase, b) at least one heat-induced gelling polymer, c) at least one water-miscible volatile solvent in a concentration ranging from 1% to 20% by weight relative to the total weight of the composition, d) at least one organic UV-screening agent. The invention relates to the use of at least one heat-induced gelling polymer and of at least one water-miscible volatile organic solvent in a cosmetic composition in the form of an oil-in-water emulsion or a water-in-oil-in-water multiple emulsion containing, in a physiologically acceptable medium, at least one agent for absorbing UV radiation, for the purpose of reducing or eliminating the effect of migration of the composition outside the application area. The invention also relates to the use of at least one heat-induced gelling polymer and of at least one water-miscible volatile organic solvent in a cosmetic composition in the form of an oil-in-water emulsion or a water-in-oil-in-water multiple emulsion containing, in a physiologically acceptable medium, at least one agent for absorbing UV radiation, for the purpose of improving the cosmetic pleasantness and the comfort of use; and more particularly for improving the spreading, improving the ocular comfort and reducing the effect of running of the composition on keratin materials.

The invention relates to cosmetic or dermatological compositions in the form of an oil-in-water or water-in-oil-in-water emulsion for protecting keratin materials, and more particularly the skin and the hair, against the harmful effects of UV radiation, containing, in a physiologically acceptable medium, at least one heat-induced gelling polymer, at least one water-miscible volatile organic solvent and at least one organic UV-screening agent.

The invention relates to the use of at least one heat-induced gelling polymer and of at least one water-miscible volatile organic solvent in a cosmetic composition in the form of an oil-in-water or water-in-oil-in-water emulsion containing, in a physiologically acceptable medium, at least one agent for absorbing UV radiation, for the purpose of reducing or eliminating the effect of migration of the composition outside the application area.

Light radiation with wavelengths of between 280 and 400 nm permits tanning of the human epidermis; rays with wavelengths of between 280 and 320 nm, which are known as UVB rays, cause skin burns and erythema that may harm the development of a tan. Rays with wavelengths of between 320 and 400 nm, which are known as UVA rays, are liable to induce impairment of the skin, especially with loss of elasticity and the appearance of wrinkles, leading to premature ageing.

UVA and UVB rays should thus be screened out, and cosmetic compositions for protecting the human epidermis containing UVA and UVB screening agents currently exist.

These photoprotective compositions are usually in the form of a composition with an aqueous continuous phase and more particularly an emulsion of oil-in-water type or of oil-in-water-in-oil-in-water multiple emulsion type, which contains, in varying concentrations, one or more standard liposoluble organic screening agents and/or standard water-soluble organic screening agents capable of selectively absorbing harmful UV radiation, these screening agents (and the amounts thereof) being selected as a function of the desired sun protection factor, the sun protection factor (SPF) being expressed mathematically as the ratio of the dose of UV radiation necessary to reach the erythema-forming threshold with the UV-screening agent to the dose of UV radiation necessary to reach the erythema-forming threshold without UV-screening agent.

The majority of these compositions are formulated in a thickened liquid form such as a milk, a cream, a cream-gel or a paste. This type of presentation is appreciated by consumers, since it facilitates the uptake of the product from its packaging without significant loss, limits the diffusion of the product to the local treatment area and allows it to be used in amounts sufficient to obtain the desired cosmetic or dermatological effect.

This property is important for photoprotective formulations that must spread uniformly on the local surface to be treated and not migrate outside this surface to be treated, which harms the overall efficacy by reducing the concentration of applied active agent. This behaviour is all the more critical when the product is subjected, during use, to a temperature increase, as in the case of antisun and photoprotective products. This temperature increase has a tendency to fluidize the product and to cause it to migrate outside the treatment area.

Furthermore, migration of the product outside the application area can cause a certain amount of inconvenience and discomfort, especially in the case of migration into the region of the eyes (stinging of the eyes and/or ocular veil) or of the lips (bitter sensation). This is likewise the case for hair compositions that must spread correctly and be distributed uniformly along the keratin fibres and not run down the forehead, the nape or the face or into the eyes.

To satisfy these conditions, thickened formulations are thus used, the viscosity of the compositions being increased by adding thickening and/or gelling polymers.

However, consumers increasingly appreciate using light cosmetic compositions that are easy to apply. Among these products, fluid compositions are, in general, more appreciated by consumers than thicker emulsions especially on account of their pleasant feel and their ease of application.

One of the major drawbacks of these compositions is that it is difficult to reconcile a pleasant feel and great ease of application with good efficacy of the final product especially while limiting the migration of the product outside the application area. The fluidity of the product favours its migration and can thus impair its efficacy and its comfort of use.

Thus, there is still a need for a wide range of antisun compositions of different viscosities, from fluid compositions to more or less thickened compositions, which are cosmetically pleasant to use and to apply, while at the same time having good cosmetic performance qualities, in terms of efficacy and staying power, i.e. non-migration of the composition outside the application area.

The Applicant has discovered, surprisingly, that antisun compositions in the form of an oil-in-water or water-in-oil-in-water emulsion containing at least one heat-induced gelling polymer and one water-miscible volatile organic solvent satisfy this need.

Thus, one subject of the present invention is a cosmetic or dermatological composition in the form of an oil-in-water emulsion or a water-in-oil-in-water multiple emulsion, characterized in that it comprises, in a physiologically acceptable medium:
a) at least one continuous aqueous phase,
b) at least one heat-induced gelling polymer,
c) at least one water-miscible volatile solvent in a concentration ranging from 1% to 20% by weight relative to the total weight of the composition,
d) at least one organic UV-screening agent.

The invention relates to the use of at least one heat-induced gelling polymer and of at least one water-miscible volatile organic solvent in a cosmetic composition in the form of an oil-in-water emulsion or a water-in-oil multiple emulsion containing, in a physiologically acceptable medium, at least one agent for absorbing UV radiation, for the purpose of reducing or eliminating the effect of migration of the composition outside the application area.

The invention also relates to the use of at least one heat-induced gelling polymer and of at least one water-miscible volatile organic solvent in a cosmetic composition in the form of an oil-in-water emulsion or a water-in-oil multiple emulsion containing, in a physiologically acceptable medium, at least one agent for absorbing UV radiation, for the purpose of improving the cosmetic pleasantness and the comfort of use; and more particularly for improving the spreading, improving the ocular comfort and reducing the effect of running of the composition on keratin materials.

The term “organic UV-screening agent” means any organic compound capable of screening out UV radiation.

The term “keratin materials” means the skin, the lips, the hair, the scalp, the eyelashes, the eyebrows and the nails.

The term “physiologically acceptable medium” means a non-toxic medium that may be applied to the skin, the lips, the hair, the eyelashes, the eyebrows and the nails. The composition of the invention may especially constitute a cosmetic or dermatological composition.

The term “water-miscible organic solvent” means any organic solvent having a solubility in water of greater than 50% by weight at 25° C.

The term “volatile organic solvent” means any organic solvent having a vapour pressure at 20° C. of greater than 17.5 mmHg.

Preferentially, the heat-induced gelling polymers in accordance with the invention are water-soluble polymers comprising water-soluble units and units having in water a lower critical solution temperature, LCST, the heat-induced demixing temperature in aqueous solution of the said units with an LCST being from 5 to 40° C. for a mass concentration in water of 1% of the said units and the concentration of the said polymer in the said composition being such that its gel point is in the range from 5 to 40° C.

The term “water-soluble polymer” generally means a polymer that is soluble in water, at a temperature of from 5 to 80° C., to a proportion of at least 10 g/l and preferably of at least 20 g/l. However, the term “water-soluble polymer” also means a polymer not necessarily having the solubility mentioned above, but which, in aqueous solution at 1% by weight, from 5 to 80° C., allows the production of a macroscopically homogeneous and transparent solution, i.e. a solution with a maximum light transmittance value, irrespective of the wavelength between 400 and 800 nm, through a sample 1 cm thick, of at least 85% and preferably of at least 90%.

The term “water-soluble units” generally means that these units are soluble in water, at a temperature of from 5 to 80° C., to a proportion of at least 10 g/l and preferably of at least 20 g/l. However, the term “water-soluble units” also means units not necessarily having the solubility mentioned above, but which, in aqueous solution at 1% by weight, from 5 to 80° C., allow the production of a macroscopically homogeneous and transparent solution, i.e. a solution with a maximum light transmittance value, irrespective of the wavelength between 400 and 800 nm, through a sample 1 cm thick, at least 85% and preferably of at least 90%. These water-soluble units have no heat-induced demixing temperature of LCST type.

In this respect, it is useful to recall that the expression “units with an LCST” preferably means units whose solubility in water is modified beyond a certain temperature. These are units with a heat-induced demixing temperature (or cloud point) defining their region of solubility in water. The minimum demixing temperature obtained as a function of the concentration of polymer consisting solely of units with an LCST is known as the “LCST” (Lower Critical Solution Temperature). For each concentration of polymer with an LCST, a heat-induced solution temperature is observed. It is higher than the LCST, which is the minimum point of the curve. Below this temperature, the polymer is soluble in water, and above this temperature, the polymer loses its solubility in water.

These units with an LCST of the polymer preferably have, according to the invention, a heat-induced demixing temperature of from 5 to 40° C. for a concentration by mass in water of 1% by weight of the said units with an LCST.

More preferentially, the heat-induced demixing temperature in aqueous solution of the units with an LCST of the polymer is from 10 to 35° C. for a concentration by mass in water of 1% of the said units with an LCST.

More preferentially, the polymer concentration is such that the gel point is in the range from 10 to 35° C.

The polymer having the structure described above with water-soluble units and specific units with an LCST defined above has in aqueous solution gelation properties beyond a critical temperature, or heat-induced gelling properties.

These heat-induced gelling properties observed beyond the demixing temperature of the chains with an LCST are described especially in the following documents:
[1] D. Hourdet et al., Polymer, 1994, Vol. 35, No. 12, pages 2624-2630.
[2] F. L'Alloret et al., Coll. Polym. Sci., 1995, Vol. 273, No. 12, pages 1163-1173.
[3] F. L'Alloret, Revue de l'Institut Français du Pétrole [Review of the French Petroleum Institute], 1997, Vol. 52, No. 2, pages 117-128.

They are due to the combination of the chains with an LCST within hydrophobic microdomains beyond their demixing temperature, thus forming crosslinking nodes between the main chains.

These gelling properties are observed when the polymer concentration is sufficient to allow interactions between units with an LCST borne by different macromolecules. The minimum concentration required, known as the “critical aggregation concentration”, or CAC, is evaluated by rheological measurements: it is the concentration at and above which the viscosity of an aqueous solution of the polymers of the invention becomes higher than the viscosity of a solution of the equivalent polymer not comprising chains with an LCST.

Beyond the CAC, the polymers of the invention have gelling properties when the temperature becomes higher than a critical value, known as the “gel point”, or T_{gel}. According to the literature data, there is good agreement between T_{gel} and the demixing temperature of the chains with an LCST, under the same concentration conditions. The gel point of an aqueous solution of a polymer of the invention is determined by rheological measurements: it is the temperature at and above which the viscosity of a solution of a polymer of the invention becomes higher than the viscosity of a solution of the equivalent polymer not comprising chains with an LCST.

The polymers of the invention are preferably characterized by a specific gel point generally of from 5 to 40° C. and preferably from 10 to 35° C., for a concentration by mass in water equal to, for example, 2% by weight.

The polymers used in the invention may be block polymers or grafted polymers, which comprise, on the one hand, water-soluble units and, on the other hand, units with an LCST as defined above.

It is pointed out that, in the present text, the water-soluble units or the units with an LCST of the polymers used according to the invention are defined as not including the groups linking together, on the one hand, the said water-soluble units and, on the other hand, the said units with an LCST.

The said linking groups are derived from the reaction, during the preparation of the polymer, of the reactive sites borne, on the one hand, by the precursors of the said water-soluble units and, on the other hand, by the precursors of the said units with an LCST.

The polymers used in the context of the invention may thus be block polymers comprising, for example, blocks consisting of water-soluble units alternating with blocks with an LCST.

These polymers may also be in the form of grafted polymers whose backbone is formed from water-soluble units, the said backbone bearing grafts consisting of units with an LCST.

The said polymers may be partially crosslinked.

The expression “water-soluble units” generally means that these units are units that are soluble in water, at a temperature of from 5 to 80° C., to a proportion of at least 10 g/l and preferably of at least 20 g/l.

However, the expression “water-soluble units” also means units not necessarily having the solubility mentioned above, but which, in aqueous solution at 1% by weight, from 5 to 80° C., allow the production of a macroscopically homogeneous and transparent solution, that is to say a solution having a maximum light transmittance value, irrespective of the wavelength between 400 and 800 nm, through a sample 1 cm thick, of at least 85% and preferably of at least 90%. These water-soluble units do not have a heat-induced demixing temperature of LCST type.

These water-soluble units may be totally or partially obtained by polymerization, especially free-radical polymerization, or by polycondensation, or may consist totally or partially of existing natural polymers or modified natural polymers.

By way of example, the water-soluble units may be totally or partially obtained by polymerization, especially free-radical polymerization, of at least one monomer chosen from the following monomers:
(meth)acrylic acid;
vinyl monomers of formula (I) below:

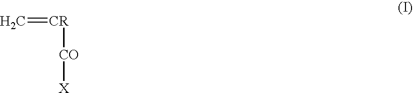

in which:
R is chosen from H, —CH₃, —C₂H₅ or —C₃H₇, and
X is chosen from:
alkyl oxides of —OR′ type in which R′ is a linear or branched, saturated or unsaturated hydrocarbon-based radical containing from 1 to 6 carbon atoms, optionally substituted with at least one halogen atom (iodine, bromine, chlorine or fluorine); a sulfonic (—SO₃⁻), sulfate (—SO₄⁻), phosphate (—PO₄H₂); hydroxyl (—OH); primary amine (—NH₂); secondary amine (—NHR₁), tertiary amine (—NR₁R₂) or quaternary amine (—N⁺R₁R₂R₃) group with R₁, R₂ and R₃ being, independently of each other, a linear or branched, saturated or unsaturated hydrocarbon-based radical containing 1 to 6 carbon atoms, with the proviso that the sum of the carbon atoms of R′+R₁+R₂+R₃ does not exceed 7; and
—NH₂, —NHR₄ and —NR₄R₅ groups in which R₄ and R₅ are, independently of each other, linear or branched, saturated or unsaturated hydrocarbon-based radicals containing 1 to 6 carbon atoms, with the proviso that the total number of carbon atoms of R₄+R₅ does not exceed 7, the said R₄ and R₅ optionally being substituted with a halogen atom (iodine, bromine, chlorine or fluorine); a hydroxyl (—OH); sulfonic (—SO₃⁻); sulfate (—SO₄⁻); phosphate (—PO₄H₂); primary amine (—NH₂); secondary amine (—NHR₁), tertiary amine (—NR₁R₂) and/or quaternary amine (—N⁺R₁R₂R₃) group with R₁, R₂ and R₃ being, independently of each other, a linear or branched, saturated or unsaturated hydrocarbon-based radical containing 1 to 6 carbon atoms, with the proviso that the sum of the carbon atoms of R₄+R₅+R₁+R₂+R₃ does not exceed 7;
maleic anhydride;
itaconic acid;
vinyl alcohol of formula CH₂═CHOH;
vinyl acetate of formula CH₂═CH—OCOCH₃;
N-vinyllactams such as N-vinylpyrrolidone, N-vinylcaprolactam and N-butyrolactam;
vinyl ethers of formula CH₂═CHOR₆ in which R₆ is a linear or branched, saturated or unsaturated hydrocarbon-based radical containing from 1 to 6 carbons;
water-soluble styrene derivatives, especially styrene sulfonate;
dimethyldiallylammonium chloride; and
vinylacetamide.

The polycondensates and natural polymers or modified natural polymers which may constitute all or part of the water-soluble units are chosen from one or more of the following components:
water-soluble polyurethanes,
xanthan gum, especially the product sold under the names Keltrol T and Keltrol SF by Kelco; or Rhodigel SM and Rhodigel 200 from Rhodia;
alginates (Kelcosol from Monsanto) and derivatives thereof such as propylene glycol alginate (Kelcoloid LVF from Kelco);
cellulose derivatives and especially carboxymethylcellulose (Aquasorb A500, Hercules), hydroxypropylcellulose, hydroxyethylcellulose and quaternized hydroxyethylcellulose;
galactomannans and derivatives thereof, such as Konjac gum, guar gum, hydroxypropyl guar, hydroxypropyl guar modified with sodium methylcarboxylate groups (Jaguar XC97-1, Rhodia), guar hydroxypropyltrimethylammonium chloride.

Mention may also be made of polyethyleneimine.

The water-soluble units preferably have a molar mass ranging from 1000 g/mol to 5 000 000 g/mol when they constitute the water-soluble backbone of a grafted polymer.

These water-soluble units preferably have a molar mass ranging from 500 g/mol to 100 000 g/mol when they constitute a block of a multiblock polymer.

The units with an LCST of the polymers used in the invention may be defined as being units whose water solubility is modified beyond a certain temperature. They are units with a heat-induced demixing temperature (or cloud point) defining their region of solubility in water. The minimum demixing temperature obtained as a function of the polymer concentration is referred to as the “LCST” (Lower Critical Solution Temperature). For each polymer concentration, a heat-induced demixing temperature is observed; it is higher than the LCST, which is the minimum point of the curve. Below this temperature, the polymer constituting the unit with an LCST is soluble in water; above this temperature, the polymer constituting the unit with an LCST loses its solubility in water.

Some of these polymers with an LCST are especially described in the following articles:
Taylor et al., Journal of Polymer Science, part A: Polymer Chemistry, 1975, 13, 2551;
J. Bailey et al., Journal of Applied Polymer Science, 1959, 1, 56;
Heskins et al., Journal of Macromolecular Science, Chemistry A2, 1968, Vol. 8, 1441.

The expression “soluble in water at a temperature T” means that the units have a solubility at T of at least 1 g/l and preferably of at least 2 g/l.

The measurement of the LCST may be performed visually: the temperature at which the cloud point of the aqueous solution appears is determined; this cloud point is reflected by the opacification of the solution, or the loss of transparency.

In general, a transparent composition will have a maximum light transmittance value, irrespective of the wavelength between 400 and 800 nm, through a sample 1 cm thick, of at least 85% and preferably of at least 90%.

The transmittance may be measured by placing a sample 1 cm thick in the light beam of a spectrophotometer working at the wavelengths of the light spectrum.

The units with an LCST of the polymers used in the invention may consist of one or more polymers chosen from the following polymers:
polyethers such as polyethylene oxide (PEO), polypropylene oxide (PPO) or random copolymers of ethylene oxide (EO) and of propylene oxide (PO),
polyvinyl methyl ethers,
polymeric and copolymeric N-substituted acrylamide derivatives containing units with an LCST, such as poly-N-isopropylacrylamide (NIPAM) and poly-N-ethylacrylamide; and
polyvinylcaprolactam and vinylcaprolactam copolymers.

Preferably, the units with an LCST consist of polypropylene oxide (PPO)ₙ where n is an integer from 10 to 50, or of random copolymers of ethylene oxide (EO) and of propylene oxide (PO), represented by the formula:

(EO)ₘ(PO)ₙ

in which m is an integer ranging from 1 to 40 and preferably from 2 to 20, and n is an integer ranging from 10 to 60 and preferably from 20 to 50.

Preferably, the molar mass of these units with an LCST is from 500 to 5300 g/mol and more preferably from 1500 to 4000 g/mol.

It has been found that the random distribution of the EO and PO units is reflected by the existence of a lower critical solution temperature, beyond which a macroscopic phase separation is observed. This behaviour is different from that of block (EO) (PO) copolymers, which form micelles beyond a critical temperature known as the micellization temperature (microscopic aggregation).

The units with an LCST may thus especially be derived from amino, especially monoamino, diamino or triamino, random copolymers of ethylene oxide and of propylene oxide. Before reaction, these polymers bear reactive sites, in this case amino groups, reacting with the reactive sites of the water-soluble polymers, for example carboxyl groups, to give the final polymer used in the invention. In the final polymer, the water-soluble units are linked to the units with an LCST via linking groups derived from the reaction of the reactive sites or groups borne, respectively, by the units with an LCST and the precursors of the water-soluble units. These linking groups will be, for example, amide, ester, ether or urethane groups.

Among these commercially available polymers with an LCST, mention may be made of the copolymers sold under the name Jeffamine by Huntsman, and especially Jeffamine XTJ-507 (M-2005), Jeffamine D-2000 and Jeffamine XTJ-509 (or T-3000).

The units with an LCST may also be derived from random EO/PO copolymers containing OH end groups, such as those sold under the name Polyglycols P41 and B11 by Clariant.

Polymeric and copolymeric N-substituted acrylamide derivatives containing units with an LCST, and also polyvinylcaprolactam and vinylcaprolactam copolymers, may also be used in the invention as units with an LCST.

As examples of polymeric and copolymeric N-substituted acrylamide derivatives containing units with an LCST, mention may be made of poly-N-isopropylacrylamide, poly-N-ethylacrylamide and copolymers of N-isopropylacrylamide (or of N-ethylacrylamide) and of a vinyl monomer chosen from the monomers having the formula (I) given above, maleic anhydride, itaconic acid, vinylpyrrolidone, styrene and its derivatives, dimethyldiallylammonium chloride, vinylacetamide, vinyl ethers and vinyl acetate derivatives.

The molar mass of these polymers is preferably from 1000 g/mol to 500 000 g/mol and preferably from 2000 to 50 000 g/mol.

These polymers may be synthesized by free-radical polymerization using a pair of initiators such as aminoethanethiol hydrochloride, in the presence of potassium persulfate, so as to obtain precursor oligomers with a reactive amino end group.

As examples of vinylcaprolactam copolymers, mention may be made of copolymers of vinylcaprolactam and of a vinyl monomer of formula (I) given above, or of a monomer chosen from maleic anhydride, itaconic acid, vinylpyrrolidone, styrene and its derivatives, dimethyldiallylammonium chloride, vinylacetamide, vinyl alcohol, vinyl acetate, vinyl ethers and vinyl acetate derivatives.

The molar mass of these vinylcaprolactam polymers or copolymers is generally from 1000 g/mol to 500 000 g/mol and preferably from 2000 to 50 000 g/mol.

These compounds may be synthesized by free-radical polymerization using a pair of initiators such as aminoethanethiol hydrochloride, in the presence of potassium persulfate, so as to obtain units with an LCST containing a reactive amino end group.

The proportion by mass of the units with an LCST in the final polymer is preferably from 5% to 70%, especially from 20% to 65% and particularly from 30% to 60% by weight relative to the final polymer.

It has been seen hereinabove that the heat-induced demixing temperature of the said units with an LCST of the polymer used in the invention is from 5 to 40° C. and preferably from 10 to 35° C., for a concentration by mass in water of 1% by weight of the said units with an LCST.

The polymers used in the context of the invention may be readily prepared by a person skilled in the art on the basis of his general knowledge, using grafting, copolymerization or coupling reaction processes.

When the final polymer is in the form of a grafted polymer, especially having a water-soluble backbone with side chains or grafts with an LCST, it is possible to prepare it by grafting units with an LCST containing at least one reactive end group or reactive site, especially an amino end group or site, onto a water-soluble polymer forming the backbone, bearing at least 10% (on a molar basis) of reactive groups such as carboxylic acid functions. This reaction may be carried out in the presence of a carbodiimide such as dicyclohexylcarbodiimide or 1-(3-dimethylaminopropyl)-3-ethylcarbodiimide hydrochloride, in a solvent such as N-methylpyrrolidone or water.

Another possibility for preparing grafted polymers consists in copolymerizing, for example, a macromonomer with an LCST (chain with an LCST described above with a vinyl end group) and a water-soluble vinyl monomer such as acrylic acid or vinyl monomers of formula (I).

When the final polymer is in the form of a block polymer, it is possible to prepare it by coupling between water-soluble units and units with an LCST, these units having complementary reactive sites at each end.

In the case of grafting processes and coupling processes, the reactive sites of the units with an LCST may be amine functions, especially monoamine, diamine or triamine functions, and OH functions. In this case, the reactive sites of the water-soluble units may be carboxylic acid functions. The groups linking the water-soluble units and the units with an LCST will thus be, for example, amide groups or ester groups.

The heat-induced gelling polymers in accordance with the invention may be chosen from those described in the following patents and patent applications: patent applications EP 1 307 501, EP 1 355 990, EP 1 355 625, FR 2 856 923, EP 1 493 774 and WO 04/006872, patents U.S. Pat. No. 6,878,754 and U.S. Pat. No. 6,689,856; patent applications EP 1 407 791, EP 1 416 044, FR 2 788 008, WO 03/008462, FR 2 694 939, EP 0 629 649, U.S. Pat. No. 6,645,476, WO 97/00275, WO 98/06438, WO98/29487, WO 98/48768, WO 98/50005, WO 00/07603, WO 02/076392, FR 2 820 976, WO 00/35961, WO 02/032560, EP 0 692 506, U.S. Pat. No. 6,870,012, WO 03/106536, WO 00/38651, WO 00/00222, WO 01/41735, US2003/0099709, GB 2 408 510.

Heat-induced gelling polymers that are particularly advantageous may be chosen from:

(1) polyurethanes comprising polyethylene oxide/polypropylene oxide/polyethylene oxide (or PEO-PPO-PEO) groups such as those described in patent applications EP-1 407 791 (Example 1 describes a polyurethane derived from the polycondensation of Pluronic F-127 with hexamethylene diisocyanate), EP-A-692 506, FR-A-2 840 907, WO 03/106 536, US-A-2005/175 573 and U.S. Pat. No. 5,702,717.

Such polyurethanes are obtained in a known manner by polycondensation of diisocyanates and of heat-sensitive PEO-PPO-PEO triblock diols and are especially described in the abovementioned patent applications.

Diisocyanates that may be mentioned include aliphatic diisocyanates, for instance ethylene diisocyanate, hexamethylene diisocyanate, decamethylene diisocyanate, and also methylene-4,4′-bis(dicyclohexyl) diisocyanate, diphenylmethane 4,4′-diisocyanate, xylylene diisocyanate, phenylene diisocyanate, tolylene diisocyanate and dimethyl diphenylene diisocyanate.

PEO-PPO-PEO triblock diols used may correspond to formula (I) below:

HO—(CH₂—CH₂—O)ₓ—(CH₂—CH(CH₃)—O)_{y}—(CH₂—CH₂—O)ₓ—H

with 20<x<120 and 20<y<120
such as the Pluronic products, especially Pluronic F-127.

The polyurethane may comprise urea and/or allophanate groups, as described in patent applications WO 03/106 536 and U.S. Pat. No. 5,702,717.

The polycondensation may also be performed in the presence of other reactive compounds, for instance diols comprising one or more carboxylic acid groups or a tertiary amine group (especially aminomethyl) or alternatively such as monohydroxylated polyethylene oxides. The polycondensation may especially be performed in the presence of water.

The polyurethane may be linear or branched.

(2) multiblock copolymers comprising a poly-N-isopropylacrylamide block and n-butyl acrylate randomly distributed and a polyethylene glycol block, such as those described in patent application EP-A-1 407 791.

The product sold under the trade name TGP-20 by the company Mebiol may be used in particular.

(3) copolymers of acrylamidomethylpropanesulfonic acid (or AMPS) such as those described in patents U.S. Pat. No. 6,645,476 and U.S. Pat. No. 6,689,856, and also the salts thereof (in particular sodium or ammonium salts) and of a macromonomer of an ester of (meth)acrylic acid and of alkoxylated C₂-C₄ alkyl (in particular ethylene oxide (EO) and/or propylene oxide (PO) (especially containing 1 to 500, more preferentially from 3 to 50 and better still 7 to 30 alkoxylated alkyl units).

Such macromonomers are especially chosen from esters of (meth)acrylic acid with an ether of polyethylene and of propylene glycol or an ether of polyglycol (8 to 25 EO) and of C10 to C22 fatty alcohol, chosen especially from Genapol C-080 or UD-080, or LA-070 or LA-110 or T-080 or T-150 or T-110 or T-200 or T-250 from Clariant.

Such macromonomers may also be derived from amino EO/PO statistical copolymers, especially mono-, di- or triamino copolymers of the Jeffamine type from Huntsman, and especially Jeffamine XTJ-507 (M-2005), Jeffamine D-2000 and Jeffamine XTJ-509 (or T-3000).

Such macromonomers may also be derived from EO/PO statistical copolymers with OH end groups, such as those sold under the name Polyglycols P41 and B11 by Clariant.

The copolymer of polyacrylamido-2-methylpropanesulfonic acid (AMPS) neutralized with aqueous ammonia (40% by weight relative to the total weight of the polymer) and of a polyether methacrylate macromonomer (60% by weight) in which the polyether is a PEO/PPO statistical copolymer comprising 5.5 mol of ethylene oxide (EO) units and 31 mol of propylene oxide units.

(4) copolymers such as those described in patent application EP 1 307 501, consisting of a polyacrylic acid (PAA) backbone bearing side chains or grafts consisting of units with an LCST chosen from:

(i) those of the type such as statistical copolymers of ethylene oxide (EO) and of propylene oxide (PO), represented by the formula:

(EO)ₘ(PO)ₙ

in which m is an integer ranging from 1 to 40 and preferably from 2 to 20, and n is an integer ranging from 10 to 60 and preferably from 20 to 50; the molar mass of these units with an LCST preferably being from 500 to 5300 g/mol and more preferentially from 1500 to 4000 g/mol;
(ii) poly-N-isopropylacrylamide polymers whose molar mass is preferably from 1000 g/mol to 500 000 g/mol and more preferentially from 2000 to 50 000 g/mol.

The content of heat-induced gelling polymer in the compositions according to the invention is preferably between 0.01% and 20% and better still between 0.1% and 10% relative to the total weight of the composition.

The water-miscible organic solvent(s) is (are) preferably chosen from C₁-C₅ lower monoalcohols such as methanol, ethanol, propanol, isopropanol, n-butanol, isobutanol and t-butanol, and more particularly ethanol.

The water-miscible volatile organic solvent(s) is (are) present at from 1% to 20% by weight and preferably in concentrations ranging from 1% to 15% and more preferentially from 1% to 10% by weight relative to the total weight of the composition.

The organic UV-screening agents are chosen especially from cinnamic derivatives; anthranilates; salicylic derivatives; dibenzoylmethane derivatives; camphor derivatives; benzophenone derivatives; diphenylacrylate derivatives; triazine derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB 2 303 549, DE 197 26 184 and EP 893 119; benzoxazole derivatives as described in patent applications EP 0 832 642, EP 1 027 883, EP 1 300 137 and DE 101 62 844; screening polymers and screening silicones such as those described especially in patent application WO 93/04665; α-alkylstyrene-based dimers, such as those described in patent application DE 198 55 649; 4,4-diarylbutadienes such as those described in patent applications EP 0 967 200, DE 197 46 654, DE 197 55 649, EP-A-1 008 586, EP 1 133 980 and EP 133 981, and mixtures thereof.

As examples of additional organic photoprotective agents, mention may be made of those denoted hereinbelow under their INCI name:

### Cinnamic Derivatives:

Ethylhexyl methoxycinnamate sold in particular under the trade name Parsol MCX by Hoffmann LaRoche,
Isopropyl methoxycinnamate,
Isoamyl methoxycinnamate sold under the trade name Neo Heliopan E 1000 by Haarmann and Reimer,

### Cinoxate,

DEA methoxycinnamate,
Diisopropyl methylcinnamate,
Glyceryl ethylhexanoate dimethoxycinnamate.

### Dibenzoylmethane Derivatives:

Butylmethoxydibenzoylmethane sold especially under the trade name Parsol 1789 by Hoffmann LaRoche,

### Isopropyldibenzoylmethane.

### Para-Aminobenzoic Acid Derivatives:

### PABA,

Ethyl PABA,

Ethyl dihydroxypropyl PABA,
Ethylhexyl dimethyl PABA sold in particular under the name Escalol 507 by ISP,

### Glyceryl PABA,

PEG-25 PABA sold under the name Uvinul P25 by BASF.

### Salicylic Derivatives:

Homosalate sold under the name Eusolex HMS by Rona/EM Industries,
Ethylhexyl salicylate sold under the name Neo Heliopan OS by Haarmann and Reimer,
Dipropylene glycol salicylate sold under the name Dipsal by Scher,
TEA salicylate sold under the name Neo Heliopan TS by Haarmann and Reimer.

### Diphenylacrylate Derivatives:

Octocrylene sold in particular under the trade name Uvinul N539 by BASF,
Etocrylene sold in particular under the trade name Uvinul N35 by BASF.

### Benzophenone Derivatives:

Benzophenone-1 sold under the trade name Uvinul 400 by BASF,
Benzophenone-2 sold under the trade name Uvinul D50 by BASF,
Benzophenone-3 or Oxybenzone sold under the trade name Uvinul M40 by BASF,
Benzophenone-4 sold under the trade name Uvinul MS40 by BASF,

### Benzophenone-5,

Benzophenone-6 sold under the trade name Helisorb 11 by Norquay,
Benzophenone-8 sold under the trade name Spectra-Sorb UV-24 by American Cyanamid,
Benzophenone-9 sold under the trade name Uvinul DS-49 by BASF,

### Benzophenone-12

n-hexyl 2-(4-diethylamino-2-hydroxybenzoyl)benzoate.

### Benzylidenecamphor Derivatives:

3-Benzylidenecamphor manufactured under the name Mexoryl SD by Chimex,
4-Methylbenzylidenecamphor sold under the name Eusolex 6300 by Merck,
Benzylidenecamphorsulfonic acid manufactured under the name Mexoryl SL by Chimex,
Camphor benzalkonium methosulfate manufactured under the name Mexoryl SO by Chimex,
Terephthalylidenedicamphorsulfonic acid manufactured under the name Mexoryl SX by Chimex,
Polyacrylamidomethylbenzylidenecamphor manufactured under the name Mexoryl SW by Chimex.

### Phenylbenzimidazole Derivatives:

Phenylbenzimidazolesulfonic acid sold in particular under the trade name Eusolex 232 by Merck,
Disodium phenyl dibenzimidazole tetrasulfonate sold under the trade name Neo Heliopan AP by Haarmann and Reimer.

### Phenylbenzotriazole Derivatives:

Drometrizole trisiloxane sold under the name Silatrizole by Rhodia Chimie,
Methylenebis(benzotriazolyl)tetramethylbutylphenol sold in solid form under the trade name MIXXIM BB/100 by Fairmount Chemical, or in micronized form as an aqueous dispersion under the trade name Tinosorb M by Ciba Specialty Chemicals.

### Triazine Derivatives:

bis-Ethylhexyloxyphenol Methoxyphenyl Triazine sold under the trade name Tinosorb S by Ciba Geigy,
Ethylhexyl triazone sold in particular under the trade name Uvinul T150 by BASF,
Diethylhexyl butamido triazone sold under the trade name Uvasorb HEB by Sigma 3V,
2,4,6-tris(diisobutyl 4′-aminobenzalmalonate)-s-triazine
the symmetrical triazine screening agents described in patent U.S. Pat. No. 6,225,467, patent application WO 2004/085 412 (see compounds 6 and 9) or the document “Symmetrical Triazine Derivatives” IP.COM Journal, IP.COM INC WEST HENRIETTA, NY, US (20 Sep. 2004), especially 2,4,6-tris(biphenyl)-1,3,5-triazines (in particular 2,4,6-tris(biphenyl-4-yl-1,3,5-triazine) and 2,4,6-tris(terphenyl)-1,3,5-triazine which is also mentioned in patent applications WO 06/035 000, WO 06/034 982, WO 06/034 991, WO 06/035 007, WO 2006/034 992 and WO 2006/034 985.

### Anthranilic Derivatives:

Menthyl anthranilate sold under the trade name Neo Heliopan MA by Haarmann and Reimer.

### Imidazoline Derivatives:

Ethylhexyldimethoxybenzylidenedioxoimidazoline propionate.

### Benzalmalonate Derivatives:

Polyorganosiloxane containing benzalmalonate functions, for instance Polysilicone-15, sold under the trade name Parsol SLX by Hoffmann LaRoche

### 4,4-Diarylbutadiene Derivatives:

1,1-Dicarboxy(2,2′-dimethylpropyl)-4,4-diphenylbutadiene

### Benzoxazole Derivatives:

2,4-Bis[5-(1-dimethylpropyl)benzoxazol-2-yl (4-phenyl)-imino]-6-(2-ethylhexyl)imino-1,3,5-triazine sold under the name Uvasorb K2A by Sigma 3V
and mixtures thereof.

The preferential additional organic photoprotective agents are chosen from:

### Ethylhexyl Methoxycinnamate,

Ethylhexyl salicylate,

### Homosalate,

### Octocrylene,

Phenylbenzimidazolesulfonic acid,

### Benzophenone-3,

### Benzophenone-4,

### Benzophenone-5,

n-Hexyl 2-(4-diethylamino-2-hydroxybenzoyl)benzoate,

### 4-Methylbenzylidenecamphor,

Terephthalylidenedicamphorsulfonic acid,
Disodium phenyldibenzimidazoletetrasulfonate,

### Methylenebis(benzotriazolyl)tetramethylbutylphenol,

bis-Ethylhexyloxyphenol Methoxyphenyl Triazine,

### Ethylhexyl Triazone,

### Diethylhexyl Butamido Triazone,

2,4,6-tris(biphenyl-4-yl)-1,3,5-triazine,
2,4,6-tris(terphenyl)-1,3,5-triazine,
Drometrizole trisiloxane,

### Polysilicone-15,

1,1-Dicarboxy(2,2′-dimethylpropyl)-4,4-diphenylbutadiene,
2,4-Bis[5-1(dimethylpropyl)benzoxazol-2-yl(4-phenyl)imino]-6-(2-ethylhexyl)imino-1,3,5-triazine,
and mixtures thereof.

The organic UV-screening agents are preferably present in the compositions according to the invention in proportions ranging from 0.01% to 20% by weight relative to the total weight of the composition and preferably ranging from 0.1% to 10% by weight relative to the total weight of the composition.

The compositions according to the invention may also comprise inorganic UV-screening agents. These agents are chosen from pigments, with a mean primary particle size generally of between 5 nm and 100 nm and preferably between 10 nm and 50 nm, of coated or uncoated metal oxides, for instance titanium oxide (amorphous or crystallized in rutile and/or anatase form), iron oxide, zinc oxide, zirconium oxide or cerium oxide pigments, which are all UV-photoprotective agents that are well known per se. Such standard coating agents are moreover alumina and/or aluminium stearate. Such coated or uncoated metal oxide pigments are described in particular in patent applications EP 518 772 and EP 518 773.

The pigments may be coated or uncoated.

The coated pigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminium salts of fatty acids, metal alkoxides (of titanium or of aluminium), polyethylene, silicones, proteins (collagen, elastin), alkanolamines, silicon oxides, metal oxides or sodium hexametaphosphate.

As is known, silicones are organosilicon polymers or oligomers of linear or cyclic, branched or crosslinked structure, of variable molecular weight, obtained by polymerization and/or polycondensation of suitably functionalized silanes, and consist essentially of a repetition of main units in which the silicon atoms are linked together via oxygen atoms (siloxane bond), optionally substituted hydrocarbon-based radicals being directly attached via a carbon atom to the said silicon atoms.

The term “silicones” also includes the silanes required for their preparation, in particular alkyl silanes.

The silicones used for coating the pigments that are suitable for the present invention are preferably chosen from the group containing alkyl silanes, polydialkylsiloxanes and polyalkylhydrogenosiloxanes. Even more preferentially, the silicones are chosen from the group containing octyltrimethylsilane, polydimethylsiloxanes and polymethylhydrogenosiloxanes.

Needless to say, before being treated with silicones, the metal oxide pigments may have been treated with other surface agents, in particular with cerium oxide, alumina, silica, aluminium compounds or silicon compounds, or mixtures thereof.

The coated pigments are more particularly titanium oxides that have been coated:
with silica, such as the product Sunveil from the company Ikeda,
with silica and iron oxide, such as the product Sunveil F from the company Ikeda,
with silica and alumina, such as the products Microtitanium Dioxide MT 500 SA and Microtitanium Dioxide MT 100 SA from the company Tayca, Tioveil from the company Tioxide and Mirasun TiW 60 from the company Rhodia,
with alumina, such as the products Tipaque TTO-55 (B) and Tipaque TTO-55 (A) from the company Ishihara and UVT 14/4 from the company Kemirai,
with alumina and aluminium stearate, such as the product Microtitanium Dioxide MT 100 T, MT 100 TX, MT 100 Z and MT-01 from the company Tayca, and the products Solaveil CT-10 W and Solaveil CT 100 from the company Uniqema, and the product Eusolex T-AVO from the company Merck,
with silica, alumina and alginic acid, such as the product MT-100 AQ from the company Tayca,
with alumina and aluminium laurate, such as the product Microtitanium Dioxide MT 100 S from the company Tayca,
with iron oxide and iron stearate, such as the product Microtitanium Dioxide MT 100 F from the company Tayca,
with zinc oxide and zinc stearate, such as the product BR351 from the company Tayca,
with silica and alumina and treated with a silicone, such as the products Microtitanium Dioxide MT 600 SAS, Microtitanium Dioxide MT 500 SAS or Microtitanium Dioxide MT 100 SAS from the company Tayca,
with silica, alumina and aluminium stearate and treated with a silicone, such as the product STT-30-DS from the company Titan Kogyo,
with silica and treated with a silicone, such as the product UV-Titan X 195 from the company Kemira,
with alumina and treated with a silicone, such as the products Tipaque TTO-55 (S) from the company Ishihara or UV Titan M 262 from the company Kemira,
with triethanolamine, such as the product STT-65-S from the company Titan Kogyo,
with stearic acid, such as the product Tipaque TTO-55 (C) from the company Ishihara,
with sodium hexametaphosphate, such as the product Microtitanium Dioxide MT 150 W from the company Tayca.

Other titanium oxide pigments treated with a silicone are preferably TiO₂ treated with octyltrimethylsilane and for which the mean size of the elementary particles is between 25 and 40 nm, such as the product sold under the trade name T 805 by the company Degussa Silices, TiO₂ treated with a polydimethylsiloxane and for which the mean size of the elementary particles is 21 nm, such as the product sold under the trade name 70250 Cardre UF TiO2SI3 by the company Cardre, anatase/rutile TiO₂ treated with a polydimethylhydrogenosiloxane and for which the mean size of the elementary particles is 25 nm, such as the product sold under the trade name Microtitanium Dioxide USP Grade Hydrophobic by the company Color Techniques.

The uncoated titanium oxide pigments are sold, for example, by the company Tayca under the trade names Microtitanium Dioxide MT 500 B or Microtitanium Dioxide MT 600 B, by the company Degussa under the name P 25, by the company Wackher under the name Transparent titanium oxide PW, by the company Myoshi Kasei under the name UFTR, by the company Tomen under the name ITS and by the company Tioxide under the name Tioveil AQ.

The uncoated zinc oxide pigments are, for example:
those sold under the name Z-Cote by the company Sunsmart;
those sold under the name Nanox by the company Elementis;
those sold under the name Nanogard WCD 2025 by the company Nanophase Technologies.

The coated zinc oxide pigments are, for example:
those sold under the name Zinc Oxide CS-5 by the company Toshibi (ZnO coated with polymethylhydrogenosiloxane);
those sold under the name Nanogard Zinc Oxide FN by the company Nanophase Technologies (as a 40% dispersion in Finsolv TN, C₁₂-C₁₅ alkyl benzoate);
those sold under the name Daitopersion ZN-30 and Daitopersion ZN-50 by the company Daito (dispersions in cyclopolymethylsiloxane/oxyethylenated polydimethylsiloxane, containing 30% or 50% of nanozinc oxides coated with silica and polymethylhydrogenosiloxane);
those sold under the name NFD Ultrafine ZnO by the company Daikin (ZnO coated with perfluoroalkyl phosphate and copolymer based on perfluoroalkylethyl as a dispersion in cyclopentasiloxane);
those sold under the name SPD-Z1 by the company Shin-Etsu (ZnO coated with silicone-grafted acrylic polymer, dispersed in cyclodimethylsiloxane);
those sold under the name Escalol 2100 by the company ISP (alumina-treated ZnO dispersed in an ethylhexyl methoxycinnamate/PVP-hexadecene/methicone copolymer mixture);
those sold under the name Fuji ZnO-SMS-10 by the company Fuji Pigment (ZnO coated with silica and polymethylsilsesquioxane);
those sold under the name Nanox Gel TN by the company Elementis (ZnO dispersed at a concentration of 55% in C₁₂-C₁₅ alkyl benzoate with hydroxystearic acid polycondensate).

The uncoated cerium oxide pigments are sold under the name Colloidal Cerium Oxide by the company Rhone-Poulenc.

The uncoated iron oxide pigments are sold, for example, by the company Arnaud under the names Nanogard WCD 2002 (FE 45B), Nanogard Iron FE 45 BL AQ, Nanogard FE 45R AQ and Nanogard WCD 2006 (FE 45R) or by the company Mitsubishi under the name TY-220,

The coated iron oxide pigments are sold, for example, by the company Arnaud under the names Nanogard WCD 2008 (FE 45B FN), Nanogard WCD 2009 (FE 45B 556), Nanogard FE 45 BL 345 and Nanogard FE 45 BL or by the company BASF under the name Transparent Iron Oxide.

Mention may also be made of mixtures of metal oxides, especially of titanium dioxide and of cerium dioxide, including the silica-coated equal-weight mixture of titanium dioxide and of cerium dioxide, sold by the company Ikeda under the name Sunveil A, and also the alumina, silica and silicone-coated mixture of titanium dioxide and of zinc dioxide, such as the product M 261 sold by the company Kemira, or the alumina, silica and glycerol-coated mixture of titanium dioxide and of zinc dioxide, such as the product M 211 sold by the company Kemira.

Treated or untreated titanium oxide particles, in amorphous form or in crystalline form (rutile and/or anatase), are most particularly preferred.

The inorganic UV-screening agents are preferably present in the compositions according to the invention in proportions ranging from 0.01% to 20% by weight relative to the total weight of the composition and preferably ranging from 0.1% to 10% by weight relative to the total weight of the composition.

The compositions are in the form of an oil-in-water emulsion (direct emulsion) or a water-in-oil-in-water emulsion (multiple emulsion).

The compositions of the invention may contain any additive usually used in cosmetics and will find applications in the fields of care, makeup and antisun products.

The compositions in accordance with the present invention may also comprise standard cosmetic adjuvants chosen especially from fatty substances, organic solvents other than water-miscible volatile organic solvents, ionic or nonionic, hydrophilic or lipophilic thickeners, softeners, humectants, opacifiers, stabilizers, emollients, silicones, antifoams, fragrances, preserving agents, anionic, cationic, nonionic, zwitterionic or amphoteric surfactants, active agents, fillers, polymers, propellants, acidifying or basifying agents or any other ingredient usually used in cosmetics and/or dermatology.

Hydrophilic thickeners that may be mentioned include carboxyvinyl polymers such as the Carbopol products (carbomers) and the Pemulen products (acrylate/C10-C30-alkylacrylate copolymer); the methacrylic acid/methyl acrylate/ethoxylated alkyl dimethyl-meta-isopropenyl benzyl isocyanate terpolymer (INCI name: Polyacrylate-3), for instance the product sold by Amerchol under the name Viscophobe DB 1000; polyacrylamides, for instance the crosslinked copolymers sold under the names Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by the company Seppic; 2-acrylamido-2-methylpropanesulfonic acid polymers and copolymers, which are optionally crosslinked and/or neutralized, for instance the poly(2-acrylamido-2-methylpropanesulfonic acid) sold by the company Clariant under the trade name Hostacerin AMPS (CTFA name: ammonium polyacryloyldimethyltauramide); cellulose derivatives such as hydroxyethylcellulose; polysaccharides and especially gums such as xanthan gum; and mixtures thereof.

Lipophilic thickeners that may be mentioned include synthetic polymers such as poly (C₁₀-C₃₀ alkyl acrylates) sold under the name Doresco IPA 13-1 by the company Landec, or modified clays such as hectorite and its derivatives, for instance the products sold under the name Bentone.

The additional organic solvents may be chosen from the group consisting of non-volatile hydrophilic organic solvents, lipophilic organic solvents and amphiphilic solvents, or mixtures thereof.

Examples of hydrophilic organic solvents that may be mentioned include polyethylene glycols containing from 6 to 80 ethylene oxides; polyols such as propylene glycol, isoprene glycol, butylene glycol, glycerol or sorbitol; monoalkyl or dialkyl isosorbides in which the alkyl groups containing from 1 to 5 carbon atoms, for instance dimethyl isosorbide; glycol ethers, for instance diethylene glycol monomethyl ether or monoethyl ether and propylene glycol ethers, for instance dipropylene glycol methyl ether.

Amphiphilic organic solvents that may be mentioned include polypropylene glycol (PPG) derivatives such as fatty acid esters of polypropylene glycol, and derivatives of PPG and of fatty alcohols, for instance PPG-23 oleyl ether, and PPG-36 oleate.

Examples of lipophilic organic solvents that may be mentioned include fatty esters such as diisopropyl adipate, dioctyl adipate or alkyl benzoates.

Preserving agents that may be mentioned include parahydroxybenzoic acid esters, also known as Parabens® (in particular methyl paraben, ethyl paraben and propyl paraben), phenoxyethanol, formaldehyde generators, for instance imidazolidinylurea or diazolidinylurea, chlorhexidine digluconate, sodium benzoate, caprylyl glycol, iodopropynyl butyl carbamate, pentylene glycol, alkyltrimethylammonium bromides such as myristyltrimethylammonium bromide (CTFA name: myrtrimonium bromide), dodecyltrimethylammonium bromide, hexadecyltrimethylammonium bromide, and mixtures thereof such as the mixture sold under the name Cetrimide® by the company FEF Chemicals. The preserving agent may be present in the composition according to the invention in a content ranging from 0.001% to 10% by weight, especially ranging from 0.1% to 5% by weight and in particular ranging from 0.2% to 3% by weight relative to the total weight of the composition.

As fillers that may be used in the composition of the invention, examples that may be mentioned include pigments; silica powder; talc; polyamide particles and especially those sold under the name Orgasol by the company Atochem; polyethylene powders; powders of natural organic materials such as starch powders, especially of crosslinked or non-crosslinked corn starch, wheat starch or rice starch, such as the starch powders crosslinked with octenylsuccinate anhydride sold under the name Dry-Flo by the company National Starch; microspheres based on acrylic copolymers, such as those made of ethylene glycol dimethacrylate/lauryl methacrylate copolymer sold by the company Dow Corning under the name Polytrap; polymethyl methacrylate powders such as those sold under the name Micropearl M 100 by the company Matsumoto; expanded powders such as hollow microspheres and especially the microspheres sold under the name Expancel by the company Kemanord Plast or under the name Micropearl F 80 ED by the company Matsumoto; silicone resin microbeads such as those sold under the name Tospearl by the company Toshiba Silicone; polyurethane powders such as the hexamethylene diisocyanate/trimethylol hexyl lactone copolymer powder sold under the name Plastic Powder D-400 by the company Toshiba Pigment (CTFA name: HDI/Trimethylol Hexyllactone Crosspolymer); and mixtures thereof. When they are present, these fillers may be in amounts ranging from 0.001% to 20% by weight, preferably from 0.1% to 10% by weight and better still from 1% to 5% by weight relative to the total weight of the composition.

Needless to say, a person skilled in the art will take care to select the optional additional compound(s) mentioned above and/or the amounts thereof such that the advantageous properties intrinsically associated with the compositions in accordance with the invention are not, or are not substantially, adversely affected by the envisaged addition(s).

Oil-in-water simple emulsions will be used more particularly.

The emulsifying surfactants are chosen in an appropriate manner depending on the chosen type of emulsion. When the emulsion is a triple emulsion, it generally comprises an emulsifier in the primary emulsion and an emulsifier in the outer phase into which the primary emulsion is introduced.

In the case of the oil-in-water simple emulsion, the dispersed oily phase may represent from 1% to 70% by weight, preferably from 5% to 60% by weight and better still from 10% to 50% by weight relative to the total weight of the simple emulsion.

In the case of the water-in-oil-in-water triple emulsion, the W/O primary emulsion may represent, for example, from 5% to 70% by weight, preferably from 10% to 60% by weight and better still from 15% to 50% by weight relative to the total weight of the triple emulsion. The inner aqueous phase of the W/O primary emulsion preferably represents from 5% to 90%, better still from 30% to 90% and even better still from 40% to 80% of the total weight of the primary emulsion.

The emulsifying surfactants are generally present in a minimum concentration of 0.5% by weight relative to the total weight of the composition.

They are preferably used in proportions ranging from 0.5% to 30% by weight, preferably from 0.5% to 20% by weight and better still from 0.5% to 15% by weight relative to the total weight of the composition.

As emulsifiers that may be used for the preparation of the O/W emulsions, examples that may be mentioned include nonionic emulsifiers such as fatty acid esters of oxyalkylenated (more particularly polyoxyethylenated) polyols, for example polyethylene glycol stearates, for instance PEG-100 stearate, PEG-50 stearate and PEG-40 stearate; fatty acid esters of oxyalkylenated sorbitan comprising, for example, from 20 to 100 EO, for example those sold under the trade names Tween 20 or Tween 60 by the company Uniqema; oxyalkylenated (oxyethylenated and/or oxypropylenated) fatty alkyl ethers; alkoxylated or non-alkoxylated sugar esters, for instance sucrose stearate such as PEG-20 methylglucose sesquistearate; sorbitan esters such as the sorbitan palmitate sold under the name Span 40 by the company Uniqema; diacid esters of fatty alcohols, for instance dimyristyl tartrate; mixtures of these emulsifiers, for instance the mixture of glyceryl stearate and of PEG-100 stearate (CTFA name: Glyceryl Stearate/PEG-100 Stearate) sold under the name Arlacel 165 by the company Uniqema and under the name Simulsol 165 by the company SEPPIC; or the mixture of dimyristyl tartrate, cetearyl alcohol, Pareth-7 and PEG-25 laureth-25, sold under the name Cosmacol PSE by the company Sasol (CTFA name: Dimyristyl tartrate/cetearyl alcohol/12-15 Pareth-7/PPG 25 laureth 25).

Co-emulsifiers such as, for example, fatty alcohols containing from 8 to 26 carbon atoms, for instance cetyl alcohol, stearyl alcohol and the mixture thereof (cetearyl alcohol), octyldodecanol, 2-butyloctanol, 2-hexyldecanol, 2-undecylpentadecanol or oleyl alcohol, or fatty acids, may be added to these emulsifiers.

In the W/O/W triple emulsions according to the invention, the W/O primary emulsion advantageously comprises at least one emulsifier with an HLB of less than 10 (HLB=Hydrophilic Lipophilic Balance). The emulsifiers may be chosen, for example, from the group comprising alkoxylated and especially ethoxylated fatty alcohols, alkoxylated and especially ethoxylated fatty esters, glycerolated esters or ethers (for instance polyglyceryl-4 isostearate), fatty acid salts such as aluminium stearate, sugar-based surfactants such as methylglucose isostearate, polyolefin-based polymeric surfactants and silicone emulsifiers, and mixtures thereof.

For the polyolefin-based polymeric surfactants, the apolar part is chosen from polyolefins such as ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene polymers and/or copolymers. The polymer chains may or may not be hydrogenated. They consist of at least 40 carbons and preferably from 60 to 700 carbons.

The polar part of these polymeric surfactants may be anionic, cationic, nonionic, zwitterionic or amphoteric. It consists, for example, of acrylic, polyalkylene glycol or polyalkyleneimine derivatives. The polymeric surfactants containing a carboxylic acid polar part are derived, for example, from the reaction of a polyolefin with carboxylic acids such as maleic acid, maleic anhydride, fumaric acid, itaconic acid, citraconic acid, mesaconic acid and aconitic acid. Preferably, the polar part consists of succinic acid or anhydride, ester or amide derivatives thereof, the corresponding salts of alkali metal, alkaline-earth metal organic ions, or alternatively of polyoxyethylene.

The polyolefin-based polymeric surfactants are also chosen from the polyolefin derivatives of succinic acid described in patents U.S. Pat. No. 4,234,435, U.S. Pat. No. 4,708,753, U.S. Pat. No. 5,129,972, U.S. Pat. No. 4,931,110, GB 2 156 799 and U.S. Pat. No. 4,919,179. The polyolefin part may consist of hydrogenated or non-hydrogenated polyisobutylene. The succinic acid or anhydride may be modified with alcohols, amines, alkanolamines or polyols, or alternatively may be in the form of alkali metal or alkaline-earth metal ions or organic ions, for instance diethanolammonium or triethanolammonium ions. Mention may be made especially of polyisobutylenes containing modified succinic end groups, such as the products sold under the names L2724, L2721, L2722, OS156565 and Lubrizol 5603 by the company Lubrizol, or Chemcinnate 2000 sold by the company Chemron. Another example of a polymeric surfactant that may be used in the invention is the product of the reaction of maleic anhydride with polyisobutylene, such as Glissopal SA sold by BASF.

The silicone emulsifiers may be chosen, for example, from the group comprising dimethicone copolyols, alkyldimethicone copolyols possibly comprising heteroatoms such as fluorine, and mixtures containing them, for example the mixture of polyglyceryl-4 isostearate/cetyl dimethicone copolyol/hexyl laurate sold under the name Abil WE 09 by the company Goldschmidt, cetyl dimethicone copolyol sold under the name Abil EM 90 by the company Goldschmidt and the mixture of cyclomethicone/dimethicone copolyol sold under the name Q2-3225C by the company Dow Corning. The emulsifier content in the primary emulsion generally ranges from 0.1% to 10% by weight and preferably from 0.5% to 5% by weight of active material relative to the total weight of the primary emulsion.

The emulsions in accordance with the invention generally comprise at least one oily phase that contains at least one oil, especially a cosmetic oil. The term “oil” means a fatty substance that is liquid at room temperature (25° C.).

As oils that may be used in the composition of the invention, it is possible to use, for example, hydrocarbon-based oils of animal origin, such as perhydrosqualene (or squalane); hydrocarbon-based oils of plant origin, such as caprylic/capric acid triglycerides, for instance those sold by the company Stearineries Dubois or those sold under the names Miglyol 810, 812 and 818 by the company Dynamit Nobel, or alternatively oils of plant origin, for instance sunflower oil, corn oil, soybean oil, marrow oil, grapeseed oil, sesame seed oil, hazelnut oil, apricot oil, macadamia oil, arara oil, coriander oil, castor oil, avocado oil, jojoba oil and shea butter oil; synthetic oils; silicone oils, for instance volatile or non-volatile polymethylsiloxanes (PDMS) containing a linear or cyclic silicone chain, which are liquid or pasty at room temperature; fluoro oils, such as partially hydrocarbon-based and/or silicone-based fluoro oils, for instance those described in document JP-A-2 295 912; ethers, such as dicaprylyl ether (CTFA name: Dicaprylyl ether); esters, for instance C₁₂-C₁₅ fatty alkyl benzoates (Finsolv TN from Finetex); arylalkyl benzoates, for instance 2-phenylethyl benzoate (X-Tend 226 from ISP); and amidated oils, for instance isopropyl N-lauroylsarcosinate (Eldew SL-205 from Ajimoto); mixtures thereof.

The oily phase may also comprise one or more fatty substances chosen, for example, from fatty alcohols (cetyl alcohol, stearyl alcohol or cetearyl alcohol), fatty acids (stearic acid) or waxes (paraffin, polyethylene wax, carnauba wax or beeswax). The oily phase may contain lipophilic gelling agents, surfactants or organic or mineral particles. The oily phase may preferably represent from 2% to 70% by weight of oil relative to the total weight of the composition.

When it is an emulsion, the aqueous phase of the said emulsion may comprise a nonionic vesicular dispersion prepared according to known processes (Bangham, Standish and Watkins, J. Mol. Biol. 13, 238 (1965), FR 2 315 991 and FR 2 416 008).

According to one particular embodiment of the invention, the oil-in-water emulsions may comprise only 1% by weight or less of emulsifying surfactants, and may even be free of emulsifying surfactants, while at the same time being stable on storage. In this case, they may be stabilized via various techniques such as the use of the hydrophilic or lipophilic thickeners such as those of patent EP 864 320, amphiphilic polymers such as those mentioned in patent EP 1 093 796 or in patent application WO 02/44231, and solid particles (Pickering-type emulsions) such as the emulsions mentioned in patent applications WO 98/42300, WO 98/42301, EP 98/7001, EP 98/7002, EP 98/7003, EP 98/7004 EP 98/7005 EP 98/7006 EP 98/7007 EP 98/7008, WO 2000/07548, WO 2000/07549 and EP 99/2233. Among the emulsion stabilizers that will be used more particularly are isophthalic acid or sulfoisophthalic acid polymers, and in particular phthalate/sulfoisophthalate/glycol copolymers (for example diethylene glycol/phthalate/isophthalate/1,4-cyclohexanedimethanol) sold under the name Eastman AQ Polymer (AQ35S, AQ38S, AQ55S and AQ48 Ultra) by the company Eastman Chemical.

Another subject of the present invention consists of the use of the compositions according to the invention as defined above for the manufacture of cosmetic products for treating the skin, the lips, the nails, the hair, the eyelashes, the eyebrows and/or the scalp, especially care products, antisun products and makeup products.

The composition according to the invention may constitute a skincare product, especially for the face, the neck, the contour of the eyes or the body; or a makeup product such as a complexion product (especially a foundation), a concealer product, an antisun product or a skin-cleansing product. The composition according to the invention will preferentially be an antisun product.

The cosmetic compositions according to the invention may be used, for example, as care and/or antisun products for the face and/or the body. They may optionally be conditioned in aerosol form and may be in the form of mousse or spray.

The compositions according to the invention in the form of vaporizable fluid lotions in accordance with the invention are applied to the skin of the hair in the form of fine particles by means of pressurization devices. The devices in accordance with the invention are well known to those skilled in the art and comprise non-aerosol pumps or “atomizers”, aerosol containers comprising a propellant and also aerosol pumps using compressed air as propellant. The latter devices are described in patents U.S. Pat. No. 4,077,441 and U.S. Pat. No. 4,850,517 (which form an integral part of the content of the description).

The compositions conditioned in aerosol form in accordance with the invention generally contain conventional propellants, for instance hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

The invention will now be described with reference to the examples that follow, which are given as non-limiting illustrations. In these examples, unless otherwise indicated, the amounts are expressed as weight percentages. The following antisun formulations were prepared; the amounts are indicated as weight percentages:

### EXAMPLES

| | % by |
|---|---|
| Composition 1 | weight |
| Silica-treated micronized titanium oxide | 5 |
| (rutile)/aluminium hydroxide/alginic acid | |
| (MT-100 AQ from the company Tayca) | |
| Preserving agents | 1 |
| 96° ethyl alcohol | 10 |
| Water | 46.19 |
| 4-tert-Butyl-4′-methoxydibenzoylmethane | 2.5 |
| (Parsol 1789 from DSM) | |
| Glycerol | 4 |
| Pentasodium salt of ethylenediaminetetra- | 0.3 |
| methylenephosphonic acid | |
| Isohexadecane | 4 |
| Terephthalylidenedicamphorsulfonic acid | 1.5 |
| manufactured under the name Mexoryl SX by | |
| Chimex | |
| Fragrance | 0.5 |
| Propylene glycol | 4 |
| Triethanolamine | 0.26 |
| Cyclohexadimethylsiloxane | 2 |
| Octocrylene sold especially under the trade | 9 |
| name Uvinul N539 by BASF | |
| Drometrizole trisiloxane | 0.75 |
| Isononyl isononanoate | 6 |
| Heat-induced gelling polyurethane comprising | 3 |
| polyethylene oxide/polypropylene oxide/polyethylene | |
| oxide groups | |

| | % by |
|---|---|
| Composition 2 | weight |
| Silica-treated micronized titanium oxide | 5 |
| (rutile)/aluminium hydroxide/alginic acid | |
| (MT-100 AQ from the company Tayca) | |
| Preserving agents | 1 |
| 96° ethyl alcohol | 10 |
| Water | 46.19 |
| 4-tert-Butyl-4′-methoxydibenzoylmethane | 2.5 |
| (Parsol 1789 from DSM) | |
| Glycerol | 4 |
| Pentasodium salt of ethylenediaminetetra- | 0.3 |
| methylenephosphonic acid | |
| Isohexadecane | 4 |
| Terephthalylidenedicamphorsulfonic acid | 1.5 |
| manufactured under the name Mexoryl SX by | |
| Chimex | |
| Fragrance | 0.5 |
| Propylene glycol | 4 |
| Triethanolamine | 0.26 |
| Cyclohexadimethylsiloxane | 2 |
| Octocrylene sold especially under the trade | 9 |
| name Uvinul N539 by BASF | |
| Drometrizole trisiloxane | 0.75 |
| Isononyl isononanoate | 6 |
| (*)Copolymer of AMPS neutralized with NH₃ | 3 |
| (40% by weight) and of a polyether | |
| methacrylate (PEO (5.5 mol)/PPO (31 mol) | |
| statistical copolymer) | |

(*)The heat-induced gelling polymer used in this example is a copolymer of polyacrylamido-2-methylpropanesulfonic acid (AMPS) neutralized with aqueous ammonia (40% by weight) and/or a polyether methacrylate macromonomer (60%) in which the polyether is a PEO/PPO statistical copolymer comprising 5.5 mol of ethylene oxide (EO) units and 31 mol of propylene oxide units.

The polyether methacrylate macromonomer is obtained according to the standard techniques by esterification of methacrylic acid and of the polyether as defined above manufactured by the company Laporte under the trade name A286.

This copolymer is obtained by precipitation polymerization in tert-butanol of AMPS and of the “polyether methacrylate” macromonomer described above according to the synthetic process described in patent application EP 1 069 142.

### Comparative Diffusion Test

| | Composition 3 | |
|---|---|---|
| | (outside the | |
| Ingredients | invention) | Composition 4 |
| Disodium EDTA | 0.1 | 0.1 |
| Triethanolamine | 0.83 | 0.83 |
| Preserving agents | 1.25 | 1.25 |
| Isohexadecane | 1 | 1 |
| Butylmethoxydibenzoylmethane | 2.5 | 2.5 |
| Terephthalylidenedicamphorsulfonic | 1.5 | 1.5 |
| acid | | |
| Octocrylene | 9 | 9 |
| Drometrizole trisiloxane | 0.75 | 0.75 |
| Titanium dioxide | 5 | 5 |
| Heat-induced gelling polyurethane | 0 | 2 |
| comprising polyethylene oxide/ | | |
| polypropylene oxide/polyethylene oxide | | |
| (or PEO-PPO-PEO) groups | | |
| Xanthan gum | 0.1 | 0.1 |
| Acrylates/C10-30 alkyl acrylate | 0.12 | 0.12 |
| crosspolymer | | |
| Dimethicone | 0.5 | 0.5 |
| Cyclopentasiloxane | 5 | 5 |
| Water | 56.85 | 54.85 |
| C₁₂-C₁₅ alkyl benzoate | 4 | 4 |
| 96° ethyl alcohol | 10 | 10 |
| Glycerol | 4 | 4 |
| Propylene glycol | 4 | 4 |
| Stearic acid | 1.5 | 1.5 |
| Glyceryl stearate (and) PEG-100 | 1 | 1 |
| stearate | | |
| Potassium cetyl phosphate | 1 | 1 |

In order to demonstrate the advantage of this combination, a diffusion test is performed according to the following protocol:

The principle of this test consists in measuring the change of the deposit of a product on absorbent paper.
a 70 g tracing paper is used
rectangles 2.5×2 cm, i.e. an area of 5 cm², are drawn on this paper
three tests are envisaged per formulation
100 mg of uniformly spread product are applied
the product is left to dry in an oven at 45° C. for 24 hours.

The sheets are removed from the oven and analysed:
when the product has diffused, a certain transparency is observed outside the application area: the periphery of this area is delimited.

The diffusion effect is observed under UV lighting using a 120 W lamp of Vilber-Lourmat type equipped with 312 and 365 nm tubes.

The sheet visualized under the full lighting of the Vilber-Lourmat lamp has the following appearance:

The following results are observed, after 24 hours, for the two compositions:

| | Appearance of the |
|---|---|
| Ingredients (INCI name) | sheet |
| Composition 3 (outside the | Strong diffusion |
| invention) | |
| Composition 4 (invention) | Absence of |
| | diffusion |

It is very clearly observed that composition 4 containing the heat-induced gelling polymer in the presence of alcohol has not at all migrated on the paper, even after staying for 24 hours in an oven at 45° C., unlike composition 1 not containing any heat-induced gelling polymer.

1-32. (canceled)

33. A photoprotective cosmetic composition, wherein said composition is in the form of an oil-in-water emulsion or a water-in-oil-in-water multiple emulsion and comprises, in a physiologically acceptable medium:
a) at least one continuous aqueous phase,
b) at least one heat-induced gelling polymer,
c) at least one water-miscible volatile solvent in a concentration ranging from 1% to 20% by weight relative to the total weight of the composition, and
d) at least one organic UV-screening agent.

34. The photoprotective cosmetic composition according to claim 33, wherein the at least one heat-induced gelling polymer is chosen from water-soluble polymers comprising water-soluble units and units having in water a lower critical solution temperature, a heat-induced demixing temperature in aqueous solution of said units with a lower critical solution temperature ranges from 5° C. to 40° C. for a mass concentration in water of 1% of said units, and
wherein the concentration of said at least one heat-induced gelling polymer in said photoprotective cosmetic composition is such that its gel point ranges from 5° C. to 40° C.

35. The photoprotective cosmetic composition according to claim 34, wherein the units with a lower critical solution temperature have a heat-induced demixing temperature ranging from 5° C. to 40° C. for a mass concentration in water of 1% by weight of said units with a lower critical solution temperature.

36. The photoprotective cosmetic composition according to claim 35, wherein the heat-induced demixing temperature in aqueous solution of the units with a lower critical solution temperature ranges from 10° C. to 35° C. for a mass concentration in water of 1% of said units with a lower critical solution temperature.

37. The photoprotective cosmetic composition according to claim 34, wherein the polymer concentration is such that the gel point ranges from 10° C. to 35° C.

38. The photoprotective cosmetic composition according to claim 34, wherein the polymer chosen from (1) block polymers comprising blocks consisting of water-soluble units alternating with blocks consisting of units with a lower critical solution temperature and (2) grafted polymers whose backbone consists of water-soluble units, said backbone bearing grafts consisting of units with a lower critical solution temperature, wherein said polymers may optionally be partially crosslinked.

39. The photoprotective cosmetic composition according to claim 34, wherein the water-soluble units may be totally or partially obtained by polymerization or by polycondensation, or wherein said water-soluble units may comprise existing natural or modified natural polymers.

40. The photoprotective cosmetic composition according to claim 39, wherein the polymerization is free-radical polymerization.

41. The photoprotective cosmetic composition according to claim 39, wherein the water-soluble units may be totally or partially obtained by polymerization of at least one monomer chosen from:
(meth) acrylic acid;
vinyl monomers of formula (I) below: 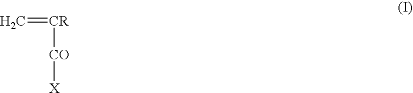
wherein:
R is chosen from H, —CH₃, —C₂H₅ or —C₃H₇, and
X is chosen from:
alkyl oxides of formula —OR′, wherein R′ is chosen from linear or branched, saturated or unsaturated hydrocarbon-based radicals comprising 1 to 6 carbon atoms, optionally substituted with at least one substituent chosen from halogens, sulfonic (—SO₃⁻) acids, sulfates (—SO₄⁻), phosphates (—PO₄H₂), hydroxyls (—OH), primary amines (—NH₂), secondary amines (—NHR₁), tertiary amines (—NR₁R₂), and quaternary amines (—N⁺R₁R₂R₃) group, wherein R₁, R₂, and R₃ are, independently of each other, chosen from linear or branched, saturated or unsaturated hydrocarbon-based radicals comprising 1 to 6 carbon atoms, with the proviso that the sum of the carbon atoms of R′+R₁+R₂+R₃ does not exceed 7; and —NH₂, —NHR₄ and —NR₄R₅ groups wherein R₄ and R₅ are, independently of each other, chosen from linear or branched, saturated or unsaturated hydrocarbon-based radicals comprising 1 to 6 carbon atoms, with the proviso that the total number of carbon atoms of R₄+R₅ does not exceed 7, said R₄ and R₅ optionally being substituted with at least one substituent chosen from halogens, hydroxyls (—OH), sulfonic (—SO₃⁻) acids, sulfates (—SO₄⁻), phosphates (—P0₄H₂), primary amines (—NH₂), secondary amines (—NHR₁), tertiary amines (—NR₁R₂) and quaternary amines (—N⁺R₁R₂R₃) group, wherein with R₁, R₂ and R₃ are, independently of each other, chosen from linear or branched, saturated or unsaturated hydrocarbon-based radicals comprising 1 to 6 carbon atoms, with the proviso that the sum of the carbon atoms of R₄+R₅+R₁+R₂+R₃ does not exceed 7;
maleic anhydride;
itaconic acid;
vinyl alcohol of formula CH₂═CHOH;
vinyl acetate of formula CH₂═CH—OCOCH₃;
N-vinyllactams;
vinyl ethers of formula CH₂═CHOR₆ wherein R₆ is chosen from linear or branched, saturated or unsaturated hydrocarbon-based radicals comprising from 1 to 6 carbons;
water-soluble styrene derivatives;
dimethyldiallylammonium chloride; and
vinylacetamide.

42. The photoprotective cosmetic composition according to claim 41, wherein the halogens are chosen from iodine, bromine, chlorine, and fluorine.

43. The photoprotective cosmetic composition according to claim 41, wherein the N-vinyllactams are chosen from N-vinylpyrrolidone, N-vinylcaprolactam, and N-butyrolactam.

44. The photoprotective cosmetic composition according to claim 41, wherein the water-soluble styrene derivatives are styrene sulfonate.

45. The photoprotective cosmetic composition according to claim 39, wherein the water-soluble units of the polymer comprise polycondensates or natural or modified natural polymers chosen from one or more of the following components:
water-soluble polyurethanes;
xanthan gum;
alginates and derivatives thereof such as propylene glycol alginate;
cellulose derivatives and especially carboxymethylcellulose, hydroxypropylcellulose, hydroxyethyl cellulose and quaternized hydroxyethylcellulose; galactomannans and derivatives thereof, such as konjac gum, guar gum, hydroxypropyl guar, hydroxypropyl guar modified with sodium methylcarboxylate groups, and guar hydroxypropyltrimethylammonium chloride; and
polyethyleneimine.

46. The photoprotective cosmetic composition according to claim 46, wherein the alginates and derivatives thereof are chosen from propylene glycol alginate.

47. The photoprotective cosmetic composition according to claim 45, wherein the cellulose derivatives are chosen from carboxymethylcellulose, hydroxypropylcellulose, hydroxyethyl cellulose, and quaternized hydroxyethylcellulose.

48. The photoprotective cosmetic composition according to claim 45, wherein the galactomannans and derivatives thereof are chosen from konjac gum, guar gum, hydroxypropyl guar, hydroxypropyl guar modified with sodium methylcarboxylate groups, and guar hydroxypropyltrimethylammonium chloride.

49. The photoprotective cosmetic composition according to claim 39, wherein the water-soluble units of the polymer have a molar mass ranging from 1,000 g/mol to 5,000,000 g/mol when they constitute the water-soluble backbone of a grafted polymer, or a molar mass ranging from 500 g/mol to 100,000 g/mol when they constitute a block of a multiblock polymer.

50. The photoprotective cosmetic composition according to claim 34, wherein the units with a lower critical solution temperature of the at least one heat-induced gelling polymer are chosen from:
polyethylene oxide, polypropylene oxide, statistical copolymers of ethylene oxide and propylene oxide;
polyvinyl methyl ethers;
N-substituted polymeric derivatives of acrylamide with a lower critical solution temperature;
copolymeric derivatives of acrylamide with a lower critical solution temperature;
polyvinylcaprolactam copolymers; and
vinylcaprolactam copolymers.

51. The photoprotective cosmetic composition according to claim 50, wherein the units with a lower critical solution temperature of the at least one heat-induced gelling polymer are chosen from polypropylene oxide (PPO)ₙ wherein n is an integer from 10 to 50 and statistical copolymers of ethylene oxide (EO) and of propylene oxide (PO) represented by the formula:
(EO)ₘ(PO)ₙ
wherein m is an integer ranging from 1 to 40, and n is an integer ranging from 10 to 60.

52. The photoprotective cosmetic composition according to claim 51, wherein m is an integer ranging from 2 to 20.

53. The photoprotective cosmetic composition according to claim 51, wherein n is an integer ranging from 20 to 50.

54. The photoprotective cosmetic composition according to claim 51, wherein the units with a lower critical solution temperature chosen from polypropylene oxide (PPO)ₙ and statistical copolymers of ethylene oxide (EO) and of propylene oxide (PO) have a molar mass ranging from 500 g/mol to 5300 g/mol.

55. The photoprotective cosmetic composition according to claim 54, wherein the units with a lower critical solution temperature chosen from polypropylene oxide (PPO)ₙ and statistical copolymers of ethylene oxide (EO) and of propylene oxide (PO) have a molar mass ranging from 1500 g/mol to 4000 g/mol.

56. The photoprotective cosmetic composition according to claim 34, wherein the units with a lower critical solution temperature of the at least one heat-induced gelling polymer are chosen from:
poly-N-isopropylacrylamides;
poly-N-ethylacrylamides;
copolymers of N-isopropylacrylamide and vinyl monomers of formula (I) below: 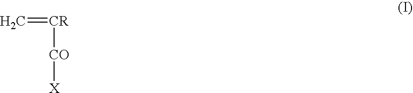
wherein:
R is chosen from H, —CH₃, —C₂H₅ or —C₃H₇, and
X is chosen from:
alkyl oxides of formula —OR′, wherein R′ is chosen from linear or branched, saturated or unsaturated hydrocarbon-based radicals comprising 1 to 6 carbon atoms, optionally substituted with at least one substituent chosen from halogens, sulfonic (—SO₃⁻) acids, sulfates (—SO₄⁻), phosphates (—PO₄H₂), hydroxyls (—OH), primary amines (—NH₂), secondary amines (—NHR₁), tertiary amines (—NR₁R₂), and quaternary amines (—N⁺R₁R₂R₃) group, wherein R₁, R₂, and R₃ are, independently of each other, chosen from linear or branched, saturated or unsaturated hydrocarbon-based radicals comprising 1 to 6 carbon atoms, with the proviso that the sum of the carbon atoms of R′+R₁+R₂+R₃ does not exceed 7; and —NH₂, —NHR₄ and —NR₄R₅ groups wherein R₄ and R₅ are, independently of each other, chosen from linear or branched, saturated or unsaturated hydrocarbon-based radicals comprising 1 to 6 carbon atoms, with the proviso that the total number of carbon atoms of R₄+R₅ does not exceed 7, said R₄ and R₅ optionally being substituted with at least one substituent chosen from halogens, hydroxyls (—OH), sulfonic (—SO₃⁻) acids, sulfates (—SO₄⁻), phosphates (—P0₄H₂), primary amines (—NH₂), secondary amines (—NHR₁), tertiary amines (—NR₁R₂) and quaternary amines (—N⁺R₁R₂R₃) group, wherein with R₁, R₂ and R₃ are, independently of each other, chosen from linear or branched, saturated or unsaturated hydrocarbon-based radicals comprising 1 to 6 carbon atoms, with the proviso that the sum of the carbon atoms of R₄+R₅+R₁+R₂+R₃ does not exceed 7;
copolymers of N-ethyl-acrylamide and vinyl monomers of formula (I) above;
maleic anhydride and derivatives thereof;
itaconic acid and derivatives thereof;
vinylpyrrolidone and derivatives thereof;
styrene and derivatives thereof;
dimethyldiallylammonium chloride;
vinylacetamide;
vinyl alcohol;
vinyl acetate and derivatives thereof; and
vinyl ethers.

57. The photoprotective cosmetic composition according to claim 56, wherein the molar mass of the units with a lower critical solution temperature of the polymer ranges from 1000 g/mol to 500,000 g/mol.

58. The photoprotective cosmetic composition according to claim 57, wherein the molar mass of the units with a lower critical solution temperature of the polymer ranges from 2000 g/mol to 50,000 g/mol.

59. The photoprotective cosmetic composition according to claim 34, wherein the mass proportion of the units with a lower critical solution temperature in the final polymer ranges from 5% to 70% by weight relative to the weight of the final polymer.

60. The photoprotective cosmetic composition according to claim 59, wherein the mass proportion of the units with a lower critical solution temperature in the final polymer ranges from 20% to 65% by weight relative to the weight of the final polymer.

61. The photoprotective cosmetic composition according to claim 60, wherein the mass proportion of the units with a lower critical solution temperature in the final polymer ranges from 30% to 60% by weight relative to weight of the final polymer.

62. The photoprotective cosmetic composition according to claim 33, wherein the at least one heat-induced gelling polymer is chosen from:
(1) polyurethanes comprising oxide/polyoxypropylene/polyethylene oxide groups;
(2) multiblock copolymers comprising at least one poly-N-isopropylacrylamide block and n-butyl acrylate randomly distributed and at least one polyethylene glycol block;
(3) copolymers of acrylamidomethylpropanesulfonic acid or the salts thereof and macromonomers of at least one ester of (meth)acrylic acid and at least one ester of ethoxylated or propoxylated alkyl; and
(4) copolymers comprising a polyacrylic acid backbone bearing at least one of sidechains and grafts consisting of units with a lower critical solution temperature chosen from:
(i) statistical copolymers of ethylene oxide and propylene oxide of the following formula:
(EO)ₘ(PO)ₙ
wherein m is an integer ranging from 1 to 40 and n is an integer ranging from 10 to 60; and wherein the molar mass of these units with a lower critical solution temperature ranges from 500 g/mol to 5300 g/mol; and
(ii) poly-N-isopropylacrylamide polymers whose molar mass ranges from 1000 g/mol to 500,000 g/mol.

63. The photoprotective cosmetic composition according to claim 62, wherein m is an integer ranging from 2 to 20.

64. The photoprotective cosmetic composition according to claim 62, wherein n is an integer ranging from 20 to 50.

65. The photoprotective cosmetic composition according to claim 62, wherein the molar mass of the units with a lower critical solution temperature ranges from 1500 to 4000 g/mol.

66. The photoprotective cosmetic composition according to claim 62, wherein poly-N-isopropylacrylamide polymers have a molar mass ranging from 2000 g/mol to 50,000 g/mol.

67. The photoprotective cosmetic composition according to claim 33, wherein the at least one heat-induced gelling polymer is present in an amount ranging from 0.01% to 20% relative to the total weight of the composition.

68. The photoprotective cosmetic composition according to claim 67, wherein the at least one heat-induced gelling polymer is present in an amount ranging from 0.1% to 10% relative to the total weight of the composition.

69. The photoprotective cosmetic composition according to claim 33, wherein the at least one water-miscible volatile solvent is chosen from C₁-C₅ lower monoalcohols.

70. The photoprotective cosmetic composition according to claim 33, wherein the at least one water-miscible volatile solvent is ethanol.

71. The photoprotective cosmetic composition according to claim 33, wherein the at least one water-miscible volatile solvent is present in an amount ranging from 1% to 15% by weight relative to the total weight of the composition.

72. The photoprotective cosmetic composition according to claim 71, wherein the at least one water-miscible volatile solvent is present in am amount ranging from 1% to 10% by weight relative to the total weight of the composition.

73. The photoprotective cosmetic composition according to claim 33, wherein the at least one organic UV-screening agent is chosen from cinnamic derivatives; anthranilates; salicylic derivatives, dibenzoylmethane derivatives, camphor derivatives; benzophenone derivatives; diphenylacrylate derivatives; triazine derivatives; benzotriazole derivatives; benzalmalonate derivatives; benzimidazole derivatives; imidazolines; bis-benzazolyl derivatives; p-aminobenzoic acid derivatives; methylenebis(hydroxyphenylbenzotriazole) derivatives; benzoxazole derivatives; screening polymers and screening silicones; alkylstyrene-based dimers; and 4,4-diarylbutadiene derivatives.

74. The photoprotective cosmetic composition according to claim 73, wherein the at least one organic UV-screening agent is chosen from:
Ethylhexyl Methoxycinnamate,
Ethylhexyl salicylate,
Homosalate,
Octocrylene,
Phenylbenzimidazolesulfonic acid,
Benzophenone-3,
Benzophenone-4,
Benzophenone-5,
n-Hexyl 2-(4-diethylamino-2-hydroxybenzoyl)benzoate,
4-Methylbenzylidenecamphor,
Terephthalylidenedicamphorsulfonic acid,
Disodium phenyldibenzimidazoletetrasulfonate,
Methylenebis(benzotriazolyl)tetramethylbutylphenol,
bis-Ethylhexyloxyphenol Methoxyphenyl Triazine,
Ethylhexyl Triazone,
Diethylhexyl Butamido Triazone,
2,4,6-tris(biphenyl-4-yl)-1,3,5-triazine,
2,4,6-tris(terphenyl)-1,3,5-triazine,
Drometrizole trisiloxane,
Polysilicone-15,
1,1-Dicarboxy(2,2′-dimethylpropyl)-4,4-diphenylbutadiene, and
2,4-Bis[5-1 (dimethylpropyl)benzoxazol-2-yl (4-phenyl)imino]-6-(2-ethylhexyl) imino-1,3,5-triazine.

75. The photoprotective cosmetic composition according to claim 33, wherein the at least one organic UV-screening agent is present in an amount ranging from 0.01% to 20% by weight relative to the total weight of the composition.

76. The photoprotective cosmetic composition according to claim 75, wherein the at least one organic UV-screening agent is present in an amount ranging from 0.1% to 10% by weight relative to the total weight of the composition.

77. The photoprotective cosmetic composition according to claim 33, further comprising at least one inorganic UV-screening agent.

78. The photoprotective cosmetic composition according to claim 77, wherein the at least one inorganic UV-screening agent is chosen from coated or uncoated metal oxide pigments whose mean primary particle size ranges from 5 nm to 100 nm.

79. The photoprotective cosmetic composition according to claim 78, wherein the mean primary particle size ranges from 10 nm to 50 nm.

80. The photoprotective cosmetic composition according to claim 77, wherein the at least one inorganic UV-screening agent is chosen from coated or uncoated titanium oxide pigments, coated or uncoated iron oxide pigments, coated or uncoated zinc oxide pigments, coated or uncoated zirconium oxide pigments, and coated or uncoated cerium oxide pigments.

82. The photoprotective cosmetic composition according to claim 77, wherein the at least one inorganic UV-screening agent is chosen from treated or untreated titanium oxide particles, in amorphous form or in crystalline form (rutile and/or anatase).

82. The photoprotective cosmetic composition according to claim 33, wherein the at least one inorganic UV-screening agent is present in an amount ranging from 0.01% to 20% by weight relative to the total weight of the composition.

83. The photoprotective cosmetic composition according to claim 82, wherein the at least one inorganic UV-screening agent is present in an amount ranging from 0.1% to 10% by weight relative to the total weight of the composition.

84. The photoprotective cosmetic composition according to claim 33, further comprising at least one cosmetic adjuvant chosen from fatty substances, organic solvents, ionic thickeners, nonionic thickeners, hydrophilic thickeners, lipophilic thickeners, softeners, humectants, opacifiers, stabilizers, emollients, silicones, antifoams, fragrances, preserving agents, anionic surfactants, cationic surfactants, nonionic surfactants, zwitterionic surfactants, amphoteric surfactants, active agents, fillers, polymers, propellants, acidifying agents, and basifying agents.
